SEQUENCE LISTING (12 sequences)

SEQ ID NO: 1 - protein, 9 aa, Artificial Sequence
  the amino acid sequence of an amino acid fragment inserted between N589 and R590 of the capsid protein VP1 of the wild-type AAV-DJ
DGTLAVPFK

SEQ ID NO: 2 - DNA, 27 bp, Artificial Sequence
  the DNA sequence encoding DGTLAVPFK and synthesized by Nanjing Genscript
gatgggactttggcggtgccttttaag

SEQ ID NO: 3 - protein, 737 aa, Artificial Sequence
  the amino acid sequence of the capsid protein VP1 of the wild-type AAV-DJ
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRLLEPLGLVEEAAKTAPGKKRPVEHSPVEPDSSSGTGKAGQQPARKRLNFGQTGDADSVPDPQPIGEPPAAPSGVGSLTMAAGGGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTTNTQTLGFSQGGPNTMANQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 4 - protein, 746 aa, Artificial Sequence
  the amino acid sequence of the capsid protein VP1 of the variant adeno-associated virus AAV-ie
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRLLEPLGLVEEAAKTAPGKKRPVEHSPVEPDSSSGTGKAGQQPARKRLNFGQTGDADSVPDPQPIGEPPAAPSGVGSLTMAAGGGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTTNTQTLGFSQGGPNTMANQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNDGTLAVPFKRQAATADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 5 - DNA, 2241 bp, Artificial Sequence
  the polynucleotide coding sequence of AAV-ie capsid VP1
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcactctcctgtggagccagactcctcctcgggaaccggaaaggcgggccagcagcctgcaagaaaaagattgaattttggtcagactggagacgcagactcagtcccagaccctcaaccaatcggagaacctcccgcagccccctcaggtgtgggatctcttacaatggctgcaggcggtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaagcaaatctccaacagcacatctggaggatcttcaaatgacaacgcctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacgacaaatacgcagactctgggcttcagccaaggtgggcctaatacaatggccaatcaggcaaagaactggctgccaggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacgatgggactttggcggtgccttttaagagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctccgcctcagatcctgatcaagaacacgcctgtacctgcggatcctccgaccaccttcaaccagtcaaagctgaactctttcatcacccagtattctactggccaagtcagcgtggagatcgagtgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 6 - DNA, 7360 bp, Artificial Sequence
  the complete sequence of the constructed Rep-Cap plasmid
gcgcgccgatatcgttaacgccccgcgccggccgctctagaactagtggatcccccggaagatcagaagttcctattccgaagttcctattctctagaaagtataggaacttctgatctgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcactctcctgtggagccagactcctcctcgggaaccggaaaggcgggccagcagcctgcaagaaaaagattgaattttggtcagactggagacgcagactcagtcccagaccctcaaccaatcggagaacctcccgcagccccctcaggtgtgggatctcttacaatggctgcaggcggtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaagcaaatctccaacagcacatctggaggatcttcaaatgacaacgcctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacgacaaatacgcagactctgggcttcagccaaggtgggcctaatacaatggccaatcaggcaaagaactggctgccaggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacgatgggactttggcggtgccttttaagagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctccgcctcagatcctgatcaagaacacgcctgtacctgcggatcctccgaccaccttcaaccagtcaaagctgaactctttcatcacccagtattctactggccaagtcagcgtggagatcgagtgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaattgcttgttaatcaataaaccgtttaattcgtttcagttgaactttggtctctgcgtatttctttcttatctagtttccatggctacgtagataagtagcatggcgggttaatcattaactacagcccgggcgtttaaacagcgggcggaggggtggagtcgtgacgtgaattacgtcatagggttagggaggtcctgtattagaggtcacgtgagtgttttgcgacattttgcgacaccatgtggtctcgctgggggggggggcccgagtgagcacgcagggtctccattttgaagcgggaggtttgaacgagcgctggcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcatttttttaaccaataggccgaaatcggcaaaatcccttataaatcaaaagaatagaccgagatagggttgagtgttgttccagtttggaacaagagtccactattaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaa

SEQ ID NO: 7 - DNA, 7330 bp, Artificial Sequence
  the plasmid sequence of AAV1
gcgcgccgatatcgttaacgccccgcgccggccgctctagaactagtggatcccccggaagatcagaagttcctattccgaagttcctattctctagaaagtataggaacttctgatctgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaattgcttgttaatcaataaaccgtttaattcgtttcagttgaactttggtctctgcgtatttctttcttatctagtttccatggctacgtagataagtagcatggcgggttaatcattaactacagcccgggcgtttaaacagcgggcggaggggtggagtcgtgacgtgaattacgtcatagggttagggaggtcctgtattagaggtcacgtgagtgttttgcgacattttgcgacaccatgtggtctcgctgggggggggggcccgagtgagcacgcagggtctccattttgaagcgggaggtttgaacgagcgctggcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcatttttttaaccaataggccgaaatcggcaaaatcccttataaatcaaaagaatagaccgagatagggttgagtgttgttccagtttggaacaagagtccactattaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaa

SEQ ID NO: 8 - DNA, 7330 bp, Artificial Sequence
  the plasmid sequence of AAV6
gcgcgccgatatcgttaacgccccgcgccggccgctctagaactagtggatcccccggaagatcagaagttcctattccgaagttcctattctctagaaagtataggaacttctgatctgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaattgcttgttaatcaataaaccgtttaattcgtttcagttgaactttggtctctgcgtatttctttcttatctagtttccatggctacgtagataagtagcatggcgggttaatcattaactacagcccgggcgtttaaacagcgggcggaggggtggagtcgtgacgtgaattacgtcatagggttagggaggtcctgtattagaggtcacgtgagtgttttgcgacattttgcgacaccatgtggtctcgctgggggggggggcccgagtgagcacgcagggtctccattttgaagcgggaggtttgaacgagcgctggcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcatttttttaaccaataggccgaaatcggcaaaatcccttataaatcaaaagaatagaccgagatagggttgagtgttgttccagtttggaacaagagtccactattaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaa

SEQ ID NO: 9 - DNA, 7330 bp, Artificial Sequence
  the plasmid sequence of AAV9
gcgcgccgatatcgttaacgccccgcgccggccgctctagaactagtggatcccccggaagatcagaagttcctattccgaagttcctattctctagaaagtataggaacttctgatctgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcgtatttctttcttatctagtttccatggctacgtagataagtagcatggcgggttaatcattaactacagcccgggcgtttaaacagcgggcggaggggtggagtcgtgacgtgaattacgtcatagggttagggaggtcctgtattagaggtcacgtgagtgttttgcgacattttgcgacaccatgtggtctcgctgggggggggggcccgagtgagcacgcagggtctccattttgaagcgggaggtttgaacgagcgctggcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcatttttttaaccaataggccgaaatcggcaaaatcccttataaatcaaaagaatagaccgagatagggttgagtgttgttccagtttggaacaagagtccactattaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaa

SEQ ID NO: 10 - DNA, 7330 bp, Artificial Sequence
  the plasmid sequence of Anc80L65
gcgcgccgatatcgttaacgccccgcgccggccgctctagaactagtggatcccccggaagatcagaagttcctattccgaagttcctattctctagaaagtataggaacttctgatctgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagcaatcaccccaggaaccagactcctcttcgggcatcggcaagaaaggccagcagcccgcgagaaagagactcaactttgggcagactggcgactcagagtcagtgcccgaccctcaaccactcggagaaccccccgcagccccctctggtgtgggatctaatacaatggctgcaggcggtggcgctccaatggcagacaataacgaaggcgccgacggagtgggtaacgcctcaggaaattggcattgcgattccacatggctgggcgacagagtcatcaccaccagcacccgaacctgggccctccccacctacaacaaccacctctacaagcaaatctccagccaatcgggaggcagcaccaacgacaacacctacttcggctacagcaccccctgggggtattttgactttaacagattccactgccacttctcaccacgtgactggcagcgactcatcaacaacaactggggattccggcccaagaagctcaacttcaagctcttcaacatccaggtcaaggaggtcacgacgaatgatggcaccacgaccatcgccaataaccttaccagcacggttcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggaatactttccttctcaaatgctgagaacgggcaacaactttcagttcagctacacgtttgaggacgtgccttttcacagcagctacgcgcacagccaaagcctggaccggctgatgaaccccctcatcgaccagtacctgtactacctgtctcggactcagaccacgagtggtaccgcaggaaatcggacgttgcaattttctcaggccgggcctagtagcatggcgaatcaggccaaaaactggctacccgggccctgctaccggcagcaacgcgtctccaagacaaccaatcaaaataacaacagcaactttgcctggaccggtgccaccaagtatcatctgaatggcagagactctctggtaaatcccggtcccgctatggcaacccacaaggacgacgaagacaaattttttccgatgagcggagtcttaatatttgggaaacagggagctggaaatagcaacgtggaccttgacaacgttatgataaccaacgaggaagaaattaaaaccaccaacccagtggccacagaagagtacggcacggtggccactaacctgcaatcggccaacaccgctcctgctacagggaccgtcaacagtcaaggagccttacctggcatggtctggcaggaccgggacgtgtacctgcagggtcctatctgggccaagattcctcacacggacggacactttcatccctcgccgctgatgggaggctttggactgaaacacccgcctcctcagatcctgattaagaatacacctgttcccgcgaatcctccaactaccttcagtccagctaagtttgcgtcgttcatcacgcagtacagcaccggacaggtcagcgtggaaattgaatgggagctgcagaaagaaaacagcaaacgctggaacccagagattcaatacacttccaactacaacaaatctacaaatgtggactttgctgttgacacaaatggcgtttattctgagcctcgccccatcggcacccgttacctcacccgtaatctgtaattgcttgttaatcaataaaccgtttaattcgtttcagttgaactttggtctctgcgtatttctttcttatctagtttccatggctacgtagataagtagcatggcgggttaatcattaactacagcccgggcgtttaaacagcgggcggaggggtggagtcgtgacgtgaattacgtcatagggttagggaggtcctgtattagaggtcacgtgagtgttttgcgacattttgcgacaccatgtggtctcgctgggggggggggcccgagtgagcacgcagggtctccattttgaagcgggaggtttgaacgagcgctggcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcatttttttaaccaataggccgaaatcggcaaaatcccttataaatcaaaagaatagaccgagatagggttgagtgttgttccagtttggaacaagagtccactattaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaa

SEQ ID NO: 11 - DNA, 5704 bp, Artificial Sequence
  the complete sequence of the genomic plasmid pAAV-CAG-mNeonGreen
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaagctttgcaaagatggataaagttttaaacagagaggaatctctcgagccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcgggggggggggggggggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatcgaattcgccaccatgggctccggagccaccaacttttctctgctgaagcaggctggcgacgtggaggagaacccaggacctatggtgtctaagggcgaggaggataacatggccagcctgcctgctacacacgagctgcacatcttcggaagcatcaacggcgtggactttgatatggtgggccagggaaccggcaacccaaacgacggatacgaggagctgaacctgaagagcacaaagggcgatctgcagttctccccctggattctggtgcctcacatcggatacggctttcaccagtacctgccatacccagacggaatgagcccattccaggctgctatggtggatggcagcggctaccaggtgcacaggaccatgcagtttgaggacggcgcctctctgaccgtgaactacagatacacatacgagggaagccacatcaagggcgaggcccaggtgaagggaaccggcttccctgctgacggaccagtgatgaccaactccctgacagccgctgactggtgccggtctaagaaaacataccctaacgataagaccatcatctctacattcaagtggagctacaccacaggaaacggcaagaggtacagatccaccgcccggaccacatacacatttgctaagccaatggccgctaactacctgaagaaccagcccatgtacgtgttccgcaagaccgagctgaagcactccaagacagagctgaacttcaaggagtggcagaaggcctttaccgacgtgatgggaatggatgagctgtacaagggatccgactacaaggaccacgatggcgactacaaggatcacgacatcgattacaaggacgatgacgataagaagcggacagctgatggcagcgagttcgagtcccccaagaagaagaggaaggtggagtgagctagcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgacttcgccttcgccctcagacgagtcggatctccctttgggccgcctccccgcagatctaacttgtttattgcagcttataatggttacaaataaagcaatagcatcacaaatttcacaaataaagcatttttttcactgcattctagttgtggtttgtccaaactcatcaatgtatcttatcatgtctggctagacacgtggccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 12 - DNA, 11635 bp, Artificial Sequence
  the complete sequence of the pHelper plasmid
ggtacccaactccatgcttaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactaggagacactttcaataaaggcaaatgtttttatttgtacactctcgggtgattatttaccccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcggggaggcggcggcgacggcgacggggacgacacgtcctccatggttggtggacgtcgcgccgcaccgcgtccgcgctcgggggtggtttcgcgctgctcctcttcccgactggccatttccttctcctataggcagaaaaagatcatggagtcagtcgagaaggaggacagcctaaccgccccctttgagttcgccaccaccgcctccaccgatgccgccaacgcgcctaccaccttccccgtcgaggcacccccgcttgaggaggaggaagtgattatcgagcaggacccaggttttgtaagcgaagacgacgaggatcgctcagtaccaacagaggataaaaagcaagaccaggacgacgcagaggcaaacgaggaacaagtcgggcggggggaccaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctgttctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcgacgaagtgccaaaaatctttgagggtcttggacgcgacgagaaacgcgcggcaaacgctctgcaacaagaaaacagcgaaaatgaaagtcactgtggagtgctggtggaacttgagggtgacaacgcgcgcctagccgtgctgaaacgcagcatcgaggtcacccactttgcctacccggcacttaacctaccccccaaggttatgagcacagtcatgagcgagctgatcgtgcgccgtgcacgacccctggagagggatgcaaacttgcaagaacaaaccgaggagggcctacccgcagttggcgatgagcagctggcgcgctggcttgagacgcgcgagcctgccgacttggaggagcgacgcaagctaatgatggccgcagtgcttgttaccgtggagcttgagtgcatgcagcggttctttgctgacccggagatgcagcgcaagctagaggaaacgttgcactacacctttcgccagggctacgtgcgccaggcctgcaaaatttccaacgtggagctctgcaacctggtctcctaccttggaattttgcacgaaaaccgcctcgggcaaaacgtgcttcattccacgctcaagggcgaggcgcgccgcgactacgtccgcgactgcgtttacttatttctgtgctacacctggcaaacggccatgggcgtgtggcagcaatgcctggaggagcgcaacctaaaggagctgcagaagctgctaaagcaaaacttgaaggacctatggacggccttcaacgagcgctccgtggccgcgcacctggcggacattatcttccccgaacgcctgcttaaaaccctgcaacagggtctgccagacttcaccagtcaaagcatgttgcaaaactttaggaactttatcctagagcgttcaggaattctgcccgccacctgctgtgcgcttcctagcgactttgtgcccattaagtaccgtgaatgccctccgccgctttggggtcactgctaccttctgcagctagccaactaccttgcctaccactccgacatcatggaagacgtgagcggtgacggcctactggagtgtcactgtcgctgcaacctatgcaccccgcaccgctccctggtctgcaattcgcaactgcttagcgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacatccttggccaattgcaagccatcaacaaagcccgccaagagtttctgctacgaaagggacggggggtttacctggacccccagtccggcgaggagctcaacccaatccccccgccgccgcagccctatcagcagccgcgggcccttgcttcccaggatggcacccaaaaagaagctgcagctgccgccgccgccacccacggacgaggaggaatactgggacagtcaggcagaggaggttttggacgaggaggaggagatgatggaagactgggacagcctagacgaagcttccgaggccgaagaggtgtcagacgaaacaccgtcaccctcggtcgcattcccctcgccggcgccccagaaattggcaaccgttcccagcatcgctacaacctccgctcctcaggcgccgccggcactgcctgttcgccgacccaaccgtagatgggacaccactggaaccagggccggtaagtctaagcagccgccgccgttagcccaagagcaacaacagcgccaaggctaccgctcgtggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccctactgcaccggcggcagcggcagcggcagcaacagcagcggtcacacagaagcaaaggcgaccggatagcaagactctgacaaagcccaagaaatccacagcggcggcagcagcaggaggaggagcgctgcgtctggcgcccaacgaacccgtatcgacccgcgagcttagaaataggatttttcccactctgtatgctatatttcaacaaagcaggggccaagaacaagagctgaaaataaaaaacaggtctctgcgctccctcacccgcagctgcctgtatcacaaaagcgaagatcagcttcggcgcacgctggaagacgcggaggctctcttcagcaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcctcgaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcgttttagggcggagtaacttgcatgtattgggaattgtagtttttttaaaatgggaagtgacgtatcgtgggaaaacggaagtgaagatttgaggaagttgtgggttttttggctttcgtttctgggcgtaggttcgcgtgcggttttctgggtgttttttgtggactttaaccgttacgtcattttttagtcctatatatactcgctctgtacttggccctttttacactgtgactgattgagctggtgccgtgtcgagtggtgttttttaataggtttttttactggtaaggctgactgttatggctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgttaaccaacctgatgtgtttaccgagtcttacattatgactccggacatgaccgaggaactgtcggtggtgctttttaatcacggtgaccagtttttttacggtcacgccggcatggccgtagtccgtcttatgcttataagggttgtttttcctgttgtaagacaggcttctaatgtttaaatgtttttttttttgttattttattttgtgtttaatgcaggaacccgcagacatgtttgagagaaaaatggtgtctttttctgtggtggttccggaacttacctgcctttatctgcatgagcatgactacgatgtgcttgcttttttgcgcgaggctttgcctgattttttgagcagcaccttgcattttatatcgccgcccatgcaacaagcttacataggggctacgctggttagcatagctccgagtatgcgtgtcataatcagtgtgggttcttttgtcatggttcctggcggggaagtggccgcgctggtccgtgcagacctgcacgattatgttcagctggccctgcgaagggacctacgggatcgcggtatttttgttaatgttccgcttttgaatcttatacaggtctgtgaggaacctgaatttttgcaatcatgattcgctgcttgaggctgaaggtggagggcgctctggagcagatttttacaatggccggacttaatattcgggatttgcttagagacatattgataaggtggcgagatgaaaattatttgggcatggttgaaggtgctggaatgtttatagaggagattcaccctgaagggtttagcctttacgtccacttggacgtgagggcagtttgccttttggaagccattgtgcaacatcttacaaatgccattatctgttctttggctgtagagtttgaccacgccaccggaggggagcgcgttcacttaatagatcttcattttgaggttttggataatcttttggaataaaaaaaaaaaaacatggttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgagctaagcgacgagaccggagacgcagatctgtttgtcacgcccgcacctggttttgcttcaggaaatatgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttccccttcgccgcccgttgagcaaccgcaagttggacagcagcctgtggctcagcagctggacagcgacatgaacttaagcgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatcaatataagctatgtggtggtggggctatactactgaatgaaaaatgacttgaaattttctgcaattgaaaaataaacacgttgaaacataacatgcaacaggttcacgattctttattcctgggcaatgtaggagaaggtgtaagagttggtagcaaaagtttcagtggtgtattttccactttcccaggaccatgtaaaagacatagagtaagtgcttacctcgctagtttctgtggattcactagaatcgatgtaggatgttgcccctcctgacgcggtaggagaaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggcccgagactcgaaccgggggtcctgcgactcaacccttggaaaataaccctccggctacagggagcgagccacttaatgctttcgctttccagcctaaccgcttacgccgcgcgcggccagtggccaaaaaagctagcgcagcagccgccgcgcctggaaggaagccaaaaggagcgctcccccgttgtctgacgtcgcacacctgggttcgacacgcgggcggtaaccgcatggatcacggcggacggccggatccggggttcgaaccccggtcgtccgccatgatacccttgcgaatttatccaccagaccacggaagagtgcccgcttacaggctctccttttgcacggtctagagcgtcaacgactgcgcacgcctcaccggccagagcgtcccgaccatggagcactttttgccgctgcgcaacatctggaaccgcgtccgcgactttccgcgcgcctccaccaccgccgccggcatcacctggatgtccaggtacatctacggattacgtcgacgtttaaaccatatgatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgatggatcc

(19) United States
(12) Patent Application Publication
(10) Pub. No.: US 2021/0355171 A1
(43) Pub. Date: Nov. 18, 2021

(54) ADENO-ASSOCIATED VIRUS HAVING A VARIANT CAPSID PROTEIN, AND USE THEREOF

(71) Applicant: SHANGHAITECH UNIVERSITY, SHANGHAI (CN)

(72) Inventors: FANGZHI TAN, SHANGHAI (CN); GUISHENG ZHONG, SHANGHAI (CN); CENFENG CHU, SHANGHAI (CN); JIEYU QI, SHANGHAI (CN)

(73) Assignee: SHANGHAITECH UNIVERSITY, SHANGHAI (CN)

(21) Appl. No.: 17/284,673

(22) PCT Filed: Sep. 24, 2019
(86) PCT No.: PCT/CN2019/107474

§ 371 Date: Apr. 12, 2021

(30) Foreign Application Priority Data
Jan. 30, 2019 (CN) 201910093000.3
Aug. 29, 2019 (CN) 201910807643.X

(51) Int. Cl. C07K 14/01; C12N 7/00; A61K 48/00

(52) U.S. Cl. CPC C07K 14/01; C12N 7/00; A61K 38/00; C12N 2750/14133; C12N 2750/14122; A61K 48/005

(57) ABSTRACT

The present disclosure provides an adeno-associated virus having a variant capsid protein, and use thereof. The variant adeno-associated virus AAV-ie refers to insert an amino acid sequence DGTLAVPFK between N589 and R590 of the capsid protein VP1 of the wild-type AAV-DJ. The variant adeno-associated virus AAV-ie can efficiently infect hair cells and supporting cells, which is greatly improved compared with the parents, therefore provides better technical support for scientific research.

### TECHNICAL FIELD

The present disclosure relates to the field of biotechnology, in particular, to an adeno-associated virus having a variant capsid protein and use thereof

### BACKGROUND

Hearing loss is one of the most common sensory disabilities, affecting more than 6.8% of the world's population (about 500 million people). The most widespread treatment for hearing loss is the use of the hearing device, which amplifies sound, enhances sound delivery, or directly stimulates neurons. This method is currently the best choice for treating hearing loss, but unfortunately, in noisy environments, the hearing sensitivity and perception of natural sounds are still limited. Therefore, better alternative strategies are needed to treat hearing loss. In recent years, gene therapy has become a promising strategy for the treatment of genetic diseases.

Half of the cases of sensorineural hearing loss are caused by genetic mutations in hair cells and supporting cells. Gene mutations in spiral ganglion neurons may also cause some hearing impairments. More than 100 mutations in deafness genes have been discovered. Hereditary hearing loss is a typical single-source disease, and a single mutation may cause hearing loss. Therefore, gene therapy is an ideal and promising potential treatment strategy that can maintain or reconstruct hearing function. For hereditary hearing loss, most deafness genes are expressed in hair cells, and mutations in some genes may affect spiral ganglion neurons. A large number of gene mutations directly affect the various functions of hair cells. However, some key deafness genes such as GJB2 are mainly expressed in supporting cells, and their mutations would affect the functions of the supporting cells, which would be accompanied by the damage to the hair cells and eventually lead to hearing loss. According to reports, there are more than 300 mutations in GJB2 that can cause genetic hearing loss. Mutations in GJB2 are the most common cause of hereditary hearing loss in humans, accounting for approximately 50% of autosomal recessive hearing loss and 15-18% of all hereditary hearing loss. Therefore, gene therapy for hereditary hearing loss needs to target both hair cells and supporting cells.

Adenovirus associated virus (AAV) is a non-enveloped icosahedral virus with a diameter of 18-26 nm. The capsid structure of AAV is composed of three capsid proteins (VP1, VP2, and VP3). The capsid contains a 4.7 kb single-stranded DNA (ssDNA), carrying two genes rep and cap, flanked by inverted terminal repeats (ITRs). Both rep and cap have multiple open reading frames (ORFs), which express the proteins needed for genome replication and packaging. Since the ITRs sequence may be directly applied to ordinary plasmids, the foreign DNA may be directly inserted into the ITRs to form a recombinant plasmid. The AAV virus gene can be cloned into a second plasmid and packaged to form a new recombinant AAV virus under the action of helper plasmid Helper. Currently, the commonly used AAVs include AAV-1, AAV-2, AAV-5, AAV-8, AAV-9 and AAV-DJ.

Several viral and non-viral gene transfer strategies have been tested in gene therapy. Among these methods, AAV-mediated gene therapy has progressed rapidly in the past decade, and more than 170 human trials (7.2%) have used AAV vectors. AAV vector has excellent safety, low immunogenicity and high transfection efficiency. The US FDA approved the first AAV-mediated gene therapy in 2017 for the treatment of rare hereditary eye diseases. However, to date, there is no gene therapy for cochlea based on AAV vectors. This may be caused by the lack of AAV with high conduction efficiency in different cell types in the cochlea. Several studies have tested AAV-mediated gene therapy for cochlea in animal models and showed promising results. However, most studies focus on hair cells and spiral ganglion neurons, due to the lack of a safe and effective AAV targeting the supporting cells. Some AAV serotypes have been evaluated in the cochlea, and all tested AAVs have a very low infection rate to the supporting cells, less than 13%. Therefore, it is necessary to design a new AAV that can effectively transfect supporting cells for the further application of gene therapy for hereditary hearing loss.

### SUMMARY

The present disclosure provides an adeno-associated virus having a variant capsid protein and use thereof, to solve the problem that the AAV in the traditional technology has a low infection rate to hair cells and supporting cells.

Based on inserting an amino acid sequence shown in SEQ ID NO. 1 between the N589 and R590 of the capsid protein VP1 of the wild-type AAV-DJ, the obtained variant AAV-ie has stronger infectivity to hair cells and supporting cells.

In one aspect, the present disclosure provides a capsid protein VP1 of a variant adeno-associated virus AAV-ie. The capsid protein VP1 of the variant adeno-associated virus AAV-ie inserts an amino acid fragment between N589 and R590 of the capsid protein VP1 of the wild-type AAV-DJ, and the amino acid sequence of the amino acid fragment is shown in SEQ ID NO. 1, specifically:

(SEQ ID NO. 1)
DGTLAVPFK

In some embodiments of the present disclosure, the amino acid sequence of the capsid protein VP1 of the wild-type AAV-DJ is shown in SEQ ID NO. 3, specifically:

(SEQ ID NO. 3)
MAADGYLPDWLEDTLSEGIRQWWKLKPGPPPPKPAERHKDDSRGLVLPGY
KYLGPFNGLDKGEPVNEADAAALEHDKAYDRQLDSGDNPYLKYNHADAEF
QERLKEDTSFGGNLGRAVFQAKKRLLEPLGLVEEAAKTAPGKKRPVEHSP
VEPDSSSGTGKAGQQPARKRLNFGQTGDADSVPDPQPIGEPPAAPSGVGS
LTMAAGGGAPMADNNEGADGVGNSSGNWHCDSTWMGDRVITTSTRTWALP
TYNNHLYKQISNSTSGGSSNDNAYFGYSTPWGYFDFNRFHCHFSPRDWQR
LINNNWGFRPKRLSFKLFNIQVKEVTQNEGTKTIANNLTSTIQVFTDSEY
QLPYVLGSAHQGCLPPFPADVFMIPQYGYLTLNNGSQAVGRSSFYCLEYF
PSQMLRTGNNFQFTYTFEDVPFHSSYAHSQSLDRLMNPLIDQYLYYLSRT
QTTGGTTNTQTLGFSQGGPNTMANQAKNWLPGPCYRQQRVSKTSADNNNS
EYSWTGATKYHLNGRDSLVNPGPAMASHKDDEEKFFPQSGVLIFGKQGSE
KTNVDIEKVMITDEEEIRTTNPVATEQYGSVSTNLQRGNRQAATADVNTQ
GVLPGMVWQDRDVYLQGPIWAKIPHTDGHFHPSPLMGGFGLKHPPPQILI
KNTPVPADPPTTFNQSKLNSFITQYSTGQVSVEIEWELQKENSKRWNPEI
QYTSNYYKSTSVDFAVNTEGVYSEPRPIGTRYLTRNL

In some embodiments of the present disclosure, the amino acid sequence of the capsid protein VP1 of the variant adeno-associated virus AAV-ie is shown in SEQ ID NO. 4, specifically:

(SEQ ID NO. 4)
MAADGYLPDWLEDTLSEGIRQWWKLKPGPPPPKPAERHKDDSRGLVLPGY
KYLGPFNGLDKGEPVNEADAAALEHDKAYDRQLDSGDNPYLKYNHADAEF
QERLKEDTSFGGNLGRAVFQAKKRLLEPLGLVEEAAKTAPGKKRPVEHSP
VEPDSSSGTGKAGQQPARKRLNFGQTGDADSVPDPQPIGEPPAAPSGVGS
LTMAAGGGAPMADNNEGADGVGNSSGNWHCDSTWMGDRVITTSTRTWALP
TYNNHLYKQISNSTSGGSSNDNAYFGYSTPWGYFDFNRFHCHFSPRDWQR
LINNNWGFRPKRLSFKLFNIQVKEVTQNEGTKTIANNLTSTIQVFTDSEY
QLPYVLGSAHQGCLPPFPADVFMIPQYGYLTLNNGSQAVGRSSFYCLEYF
PSQMLRTGNNFQFTYTFEDVPFHSSYAHSQSLDRLMNPLIDQYLYYLSRT
QTTGGTTNTQTLGFSQGGPNTMANQAKNWLPGPCYRQQRVSKTSADNNNS
EYSWTGATKYHLNGRDSLVNPGPAMASHKDDEEKFFPQSGVLIFGKQGSE
KTNVDIEKVMITDEEEIRTTNPVATEQYGSVSTNLQRGNDGTLAVPFKRQ
AATADVNTQGVLPGMVWQDRDVYLQGPIWAKIPHTDGHFHPSPLMGGFGL
KHPPPQILIKNTPVPADPPTTFNQSKLNSFITQYSTGQVSVEIEWELQKE
NSKRWNPEIQYTSNYYKSTSVDFAVNTEGVYSEPRPIGTRYLTRNL

In another aspect, the present disclosure provides an isolated nucleic acid containing the nucleotide sequence encoding the above-mentioned capsid protein VP1 of the variant adeno-associated virus AAV-ie.

In another aspect, the present disclosure provides a construct containing the above-mentioned isolated nucleic acid. The construct may generally be obtained by inserting the above-mentioned isolated nucleic acid into a suitable expression vector. Those skilled in the art may select a suitable expression vector. For example, the expression vector may be Rep2-Capsid and the like.

In another aspect, the present disclosure provides a host cell, containing the above-mentioned construct or incorporating the above-mentioned exogenous isolated nucleic acid in the genome, or containing the above-mentioned variant adeno-associated virus AAV-ie. The host cell may be a eukaryotic cell and/or a prokaryotic cell, specifically, a mouse cell, a human cell, etc., more specifically, a human embryonic kidney cell, etc., and more specifically, a HEK293FT cell.

In another aspect, the present disclosure provides a variant adeno-associated virus AAV-ie, including the above-mentioned capsid protein VP1 of the variant adeno-associated virus AAV-ie. That is, an amino acid fragment as shown in SEQ ID NO. 1 is inserted between N589 and R590 of the capsid protein VP1 of the wild-type AAV-DJ.

Further, the variant adeno-associated virus AAV-ie further includes a heterologous nucleotide sequence encoding a target product. The heterologous nucleotide sequence encoding the target product may be carried by various capsid proteins. The heterologous nucleotide sequence encoding the target product may generally be a construct, which may generally contain a nucleic acid encoding the target product. The construct may generally be obtained by inserting the nucleic acid encoding the target product into a suitable expression vector. Those skilled in the art may select a suitable expression vector. For example, the above expression vector may include, but not limited to, pAAV-CAG, pAAV-TRE, pAAV-EF1a, pAAV-GFAP promoter, pAAV-Lgr5 promoter, pAAV-Sox2 promoter and the like.

Further, the target product is a nucleic acid or a protein, and the nucleic acid may be small guide RNA (sgRNA), interfering RNA (RNAi), etc.

The variant adeno-associated virus AAV-ie may serve as a carrier material to introduce exogenous genes into the cells of the subject. Compared with the parental wild-type AAV-DJ, the infection rate of the variant adeno-associated virus AAV-ie to hair cells and supporting cells has significantly increased.

In another aspect, the present disclosure provides a pharmaceutical composition, including the variant adeno-associated virus AAV-ie as described above and pharmaceutically acceptable excipients.

In another aspect, the present disclosure provides the use of the variant adeno-associated virus AAV-ie for delivering a target product to hair cells and/or supporting cells of an individual. The delivery of the target product may be for non-diagnostic/therapeutic purposes, for example, it may be in vitro to deliver the target product to the isolated hair cells and/or supporting cells. The hair cells generally include outer hair cells and/or inner hair cells.

Further, the target product is a nucleic acid or a protein, and the nucleic acid may be small guide RNA (sgRNA), interfering RNA (RNAi), etc.

In another aspect, the present disclosure provides the use of the variant adeno-associated virus AAV-ie in the preparation of drugs for the treatment of a hearing impairment disease caused by cochlear injury in an individual.

Further, the hearing impairment disease is a disease related to hair cells and/or supporting cells and/or spiral neuron cells.

Further, the hearing impairment disease is a disease related to genetic defects, environmental damage or aging, for example, a related disease caused by gene mutation, for another example, a related disease caused by noise or drugs, for yet another example, a related disease caused by aging.

Further, the hearing impairment disease may be a disease related to cell damage, specifically cochlear hair cell damage and supporting cell damage, and more specifically, cochlear hair cell damage caused by gene mutation, supporting cell damage caused by gene mutation, cell damage caused by noise, cell damage caused by drugs, or aging.

Further, the variant adeno-associated virus AAV-ie serves as a carrier for delivering the target product. As described above, the adeno-associated virus having a variant capsid protein and use thereof of the present disclosure have the following beneficial effects:

The variant adeno-associated virus AAV-ie has higher infectivity to hair cells and supporting cells, can efficiently infect hair cells and supporting cells, and can also efficiently infect spiral ganglion neurons, which is greatly improved compared with the parents, therefore provides better technical support for scientific research.

### BRIEF DESCRIPTION OF THE DRAWINGS

FIG. 1a shows the expression of green fluorescent protein mNeonGreen after the wild-type AAV-DJ infects HEK 293T cells in Embodiment 1.

FIG. 1b shows the expression of green fluorescent protein mNeonGreen after the variant AAV-ie infects HEK 293T cells in Embodiment 1.

FIG. 1c shows the statistics of the fluorescence ratio of HEK 293T cells infected by wild-type AAV-DJ and variant AAV-ie in Embodiment 1.

FIG. 2a shows the immunostaining photographs of green fluorescence mNeonGreen and supporting cell specific marker protein Sox2 after the AAV1, AAV6, AAV9, AAV-DJ, AAV-ie and Anc80L65 infects the in-vitro cultured cochlear tissue in vitro in Embodiment 2.

FIG. 2b shows the statistical results of the data in FIG. 2a.

FIG. 3a shows the immunostaining photographs of green fluorescence mNeonGreen and hair cell specific marker protein Myo7a after the AAV1, AAV6, AAV9, AAV-DJ, AAV-ie and Anc80L65 infects the in-vitro cultured cochlear tissue in vitro in Embodiment 2.

FIG. 3b shows the statistical results of the data in FIG. 3a.

FIG. 4a shows the expression of green fluorescence mNeonGreen and the staining results of the supporting cell marker protein Sox2 represented by magenta in three areas of the cochlea in the large field of view in Embodiment 3: apex, middle and base. 14 days after the AAV-ie virus is injected into the round window of newborn mice, the cochlea is dissected out and immunostained by supporting cell specific marker protein Sox2, and then the results of green fluorescence mNeonGreen and immunostaining are photographed.

FIG. 4b shows the immunostaining photographs of green fluorescence mNeonGreen and Sox2-positive supporting cell after the AAV1, AAV6, AAV8, AAV9, DJ8, Anc80L65, AAV-DJ and AAV-ie infects the cochlear tissue in Embodiment 3.

FIG. 4c shows the statistical results of the data in FIG. 4b.

FIG. 5a shows the expression of green fluorescence mNeonGreen and the staining results of the hair cell marker protein Myo7a represented by magenta in AAV-DJ and AAV-ie in Embodiment 3.

FIG. 5b shows the statistical results of the data in FIG. 5a.

FIG. 6a shows the expression of green fluorescence mNeonGreen and the staining results of the spiral ganglion neuron marker protein NeurN represented by magenta in three areas: apex, middle and base.

FIG. 6b shows the statistical results in FIG. 6a.

FIG. 7a shows that AAV-9, PHP.eB, AAV-DJ, and AAV-ie infects Sox2-positive cochlear supporting cells to different degrees in Embodiment 4, magenta refers to Sox2-positive supporting cells, and green refers to the green fluorescent protein mNeonGreen expressed in the AAV-introduced cells.

FIG. 7b shows the statistical results of the data in FIG. 7b.

FIG. 8 shows the immunostaining results of mouse cochlear samples in Embodiment 5.

FIG. 9a shows the immunostaining results of mouse utricle samples in Embodiment 6.

FIG. 9b shows the immunostaining results of human utricle samples in Embodiment 6.

FIG. 9c is an enlarged view of FIG. 9b.

FIG. 10a shows fluorescence photographs of the cochlea injected with AAV-ie in Embodiment 7.

FIG. 10b shows scanning electron microscope images of the cochlea infected with AAV-ie in Embodiment 7.

FIG. 10c shows the enlarged view of FIG. 10b.

FIG. 10d shows the statistical results of the cells in FIG. 10b.

FIG. 10e shows the detecting results of the hearing of mice using the ABR audiometry technology in Embodiment 7.

### DETAILED DESCRIPTION OF THE PREFERRED EMBODIMENTS

The embodiments of the present disclosure will be described below through exemplary embodiments. Those skilled in the art can easily understand other advantages and effects of the present disclosure according to contents disclosed by the specification. The present disclosure can also be implemented or applied through other different exemplary embodiments. Various modifications or changes can also be made to all details in the specification based on different points of view and applications without departing from the spirit of the present disclosure.

Before further describing the specific embodiments of the present disclosure, it is understood that the scope of the present disclosure is not limited to the specific embodiments described below; It is also to be understood that the terminology of the disclosure is used to describe the specific embodiments, and not to limit the scope of the disclosure; In the present specification and claims, the singular forms “a”, “an” and “the” include the plural forms, unless specifically stated otherwise.

When the numerical values are given by the embodiments, it is to be understood that the two endpoints of each numerical range and any one between the two may be selected unless otherwise stated. Unless otherwise defined, all technical and scientific terms used in the present disclosure have the same meaning as commonly understood by one skill in the art. In addition to the specific method, equipment and material used in the embodiments, any method, equipment and material in the existing technology similar or equivalent to the method, equipment and material mentioned in the embodiments of the present disclosure may be used to realize the invention according to the grasp of the existing technology and the record of the invention by those skilled in the art.

Unless otherwise stated, the experimental methods, detection methods, and preparation methods disclosed in the present invention all employ conventional techniques of molecular biology, biochemistry, chromatin structure and analysis, analytical chemistry, cell culture, recombinant DNA technology in the technical field and related fields. These techniques are well described in the prior literature. For details, see Sambrook et al. MOLECULAR CLONING: A LABORATORY MANUAL, Second edition, Cold Spring Harbor Laboratory Press, 1989 and Third edition, 2001; Ausubel et al, CURRENT PROTOCOLS IN MOLECULAR BIOLOGY, John Wiley & Sons, New York, 1987 and periodic updates; The series METHODS IN ENZYMOLOGY, Academic Press, San Diego; Wolfe, CHROMATIN STRUCTURE AND FUNCTION, Third edition, Academic Press, San Diego, 1998; METHODS IN ENZYMOLOGY, Vol. 304, Chromatin (P. M. Wassarman and A. P. Wolffe, eds.), Academic Press, San Diego, 1999; and METHODS IN MOLECULAR BIOLOGY, Vol. 119, Chromatin Protocols (P. B. Becker, ed.) Humana Press, Totowa, 1999, and the like.

### Materials and Sources:

Young mice: Shanghai Lingchang Biological Technology Co., Ltd.

Various AAVs: synthesized and constructed by Nanjing Genscript Co., Ltd.

Donkey serum: Shanghai Yeasen Biotechnology Co., Ltd.

Antibodies and dilution ratio of the antibodies when staining:

Primary antibody: myosin 7A (Myo7a, #25-6790 Proteus Biosciences, 1:1000), Sox2 (Sox-2, #sc-17320, Santa Cruz Biotechnology, 1:1000), Flag (Flag, #F3165, Sigma Aldrich, 1:1000), NeuN (NeuN, #12943S, Cell Signaling Technology, 1:500).

Secondary antibody: the secondary antibodies of anti-rat, mouse, rabbit and goat labeled with three different colors (TRIC, FITC, Cy5) were from Invitrogen.

Reagents for cell and tissue culture: DMEM (Hyclone), fetal bovine serum (Lensa), supplement N2 (ThermoFisher), ampicillin (ThermoFisher), double antibody (ThermoFisher).

Consumables for cell and tissue culture: commonly used consumables including various culture dishes, centrifuge tubes, pipettes and disposable filters were purchased from Corning.

### Embodiment 1 Construction of AAV Variants and Infection of HEK 293T Cells

### 1. Construction of AAV Variant (Named AAV-ie) Rep-Cap Plasmid

AAV packaging requires three plasmids: genomic plasmid containing the target gene, Rep-Cap plasmid and Helper plasmid.

The sequence of the Cap protein in the Rep-Cap plasmid determines the different serotypes of AAV, which in turn affects the preference of AAV to infect cells. Therefore, a new AAV type can be obtained by modifying the Cap protein.

### (1) Construction, Enzyme Digestion and Purification of Vector

The Rep-Cap plasmid of the parental AAV-DJ was synthesized by Nanjing Genscript Co., Ltd. (www.genscript.com.cn).

First, a unique NheI restriction enzyme site was introduced between amino acids 589 and 590 of the VP1 capsid protein of the wild-type AAV-DJ by polymerase chain reaction (PCR) mutagenesis, the primer was synthesized by Nanjing Genscript Co., Ltd. (www.genscript.com.cn). Then the obtained VP1 capsid protein was digested with restriction enzyme NheI and recovered (Axygen: AP-GX-250G). The recovered fragments were tested for concentration by Nanodrop 2000.

The DNA sequence (SEQ ID NO. 2: gatgggactttggcggtgccllllaag) encoding DGTLAVPFK was synthesized by Nanjing Genscript Co., Ltd. (www.genscript.com.cn). The synthesized fragments were dissolved to 10 μM with ultrapure water.

### (2) Vector Connection, Transformation and Plasmid Extraction

The recovered backbone vector and the DNA sequence fragment encoding DGTLAVPFK were recombined and connected (Novoprotein: NR001A), the connection system is as follows: recombinant connection buffer 2 μL, backbone vector 30 ng, DNA fragment encoding DGTLAVPFK 1 μL, recombinant ligase 0.5 μL, filling ddH₂O to 10 μL, reacting at 50° C. for 20 minutes.

The transformation steps are as follows: taking 100 μL competent cells (TransGen: CD201), thawing on ice; mixing 10 μL of ligation product with the competent cells, then placing on ice for 20 minutes; heat shocking at 42° C. for 60 seconds; placing on ice for 2 minutes, adding 400 μL of resuscitation LB medium (MDBio: L001-1 kg), placing on a shaker for 30 minutes; taking 70 μL of the shaken medium, spreading on an ampicillin plate (50 μg/ml, 37° C. incubator, and incubating for 14 hours).

Selecting the monoclonal bacteria, expanding the culture in 4 ml liquid LB medium, and extracting the plasmid after 14 hours (Axygene: AP-MN-P-250G).

The steps are as follows: centrifuging the bacterial solution at 4000 rpm for 10 minutes, and discarding the supernatant medium; adding 350 μL of buffer S1, blowing away the bacteria, and transferring to a 2 ml centrifuge tube; adding 250 μL of buffer S2, inverting upside down for 8 times; adding 250 μL of buffer S3, mixing upside down for 6 times, and producing a precipitate; centrifuging at 12000 rpm for 10 minutes, obtaining the supernatant and passing through a column; centrifuging for 1 minute, discarding the waste liquid, adding 500 μL of W1, centrifuging for 1 minute, and discarding the waste liquid; adding 750 μL of W2, centrifuging, and discarding the supernatant; adding 500 μL of W2, centrifuging, and discarding the supernatant; idling for 1 minute; adding 50 μL of eluent, settling for 2 minutes, and centrifuging. After concentration detection, 10 μL of the plasmid was taken for sequencing, and the positive plasmid was stored at −20° C. The sequencing results show that the obtained plasmid is capable of encoding the variant capsid protein VP1.

Further experimental results show that the prepared plasmid is capable of expressing the variant capsid protein VP1. The polynucleotide coding sequence of AAV-ie capsid VP1 is shown in SEQ ID NO. 5. The complete sequence of the constructed Rep-Cap plasmid is shown in SEQ ID NO. 6

### 2. Packaging and Purification of AAV Variant (Named AAV-ie) Virus

The obtained Rep-Cap plasmid, a genomic plasmid pAAV-CAG-mNeonGreen (the complete sequence of the plasmid is shown in SEQ ID NO. 11) expressing a green fluorescent protein mNeonGreen (GenBank: LC279210.1), and a pHelper plasmid (the complete sequence of the plasmid is shown in SEQ ID NO. 12) were co-transfected into HEK-In 293T cells at an appropriate amount. The AAV virus was purified by ultra-high-speed centrifugation with iododiol gradient. The virus titer was measured at a suitable concentration of 1E+12-1E+13 GC/mL, and placed at −80° C. for use.

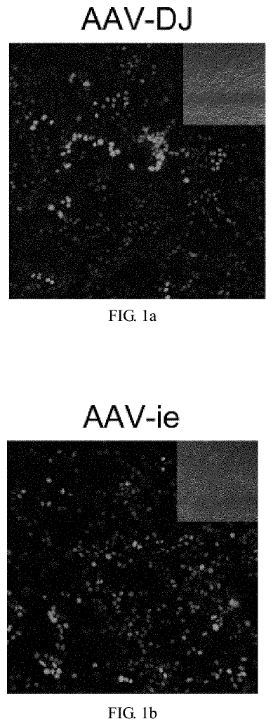

AAV viruses (AAV-ie and AAV-DJ, respectively) produced by the capsid protein variant and parental capsid protein were added to HEK 293T cells which were cultured with DMEM+10% fetal bovine serum. The MOI value of the added virus is 1000. After 48 hours, the expression of green fluorescent protein mNeonGreen was observed by a fluorescence microscope, as shown in FIGS. 1a-1c. FIGS. 1a and 1b show the signal of the green fluorescent protein, and the upper right corner shows the cells under bright field natural light.

The results show that AAV variants and their parental AAV infect HEK 293T cells at a similar ratio.

### Embodiment 2 Infections of Mouse Cochlear Tissue In Vitro by AAV Variants

Quickly removing the cochlea of P3 wild-type C57 mice (Shanghai Lingchang Biological Technology Co., Ltd.) on the ice, attaching the cochlea to a slide coated with cell-tak, and placing the slide in 98% DMEM+1% N₂+1% Amp (5 μg/mL) medium for stabilizing for 12 h, and adding 1% FBS and 2×10¹⁰ GC AAV to culture for 48-60 h. Identifying the cultured samples by immunostaining. The samples were immersed in 4% Paraformaldehyde (PFA) for fixation, and immersed in phosphate buffer saline (PBS) containing 10% donkey serum and 0.3% Triton X-100. After incubating at room temperature for 1 hour, adding the antibodies of Myo7a (myosin 7a) and Sox2 protein and the corresponding secondary antibodies. The samples were mounted with an anti-fluorescence quenching agent mounting medium, and observed with confocal microscopy.

When shooting images by the confocal method, the laser power setting for shooting variant-infected samples was selected as the standard. All visible green fluorescent protein mNeonGreen signals were captured with the same laser settings.

In terms of data processing, by calculating the numbers of mNeonGreen-positive supporting cells and hair cells, the percentage of mNeonGreen on the cochlea was manually quantified. The cells with Myo7a-positive are hair cells, and the cells with Sox2 protein-positive are supporting cells.

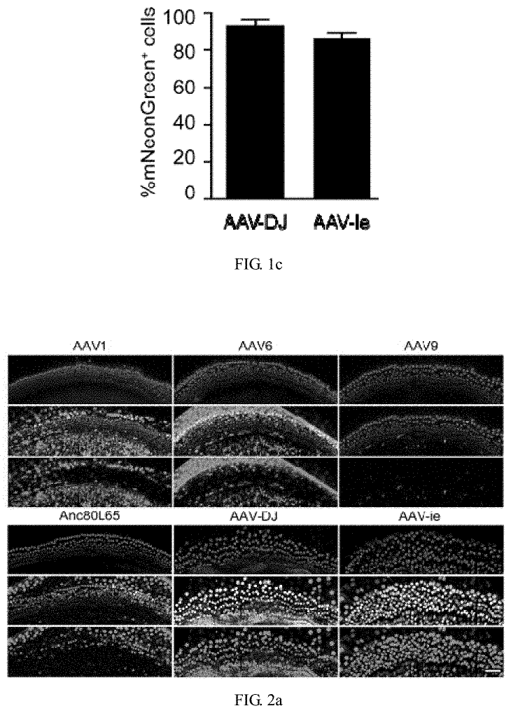

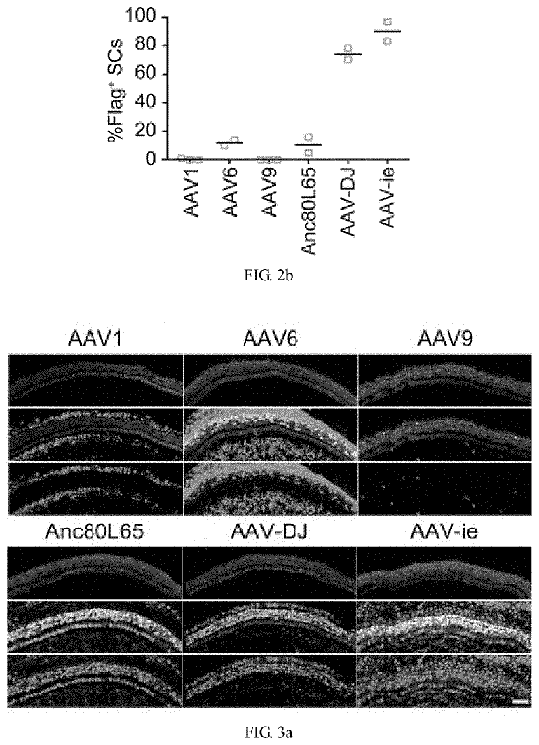

As shown in FIGS. 2a-2b, magenta represents the supporting cells, green represents the green fluorescent protein mNeonGreen expressed in the AAV-introduced cells, and FIG. 2b shows the statistical data of FIG. 2a. The results show that the AAV variant AAV-ie can efficiently infect the supporting cells of the in-vitro cultured mouse cochlear tissue. Compared with other AAV and the parental AAV, the proportion of Sox2-positive cells infected by AAV-ie is higher.

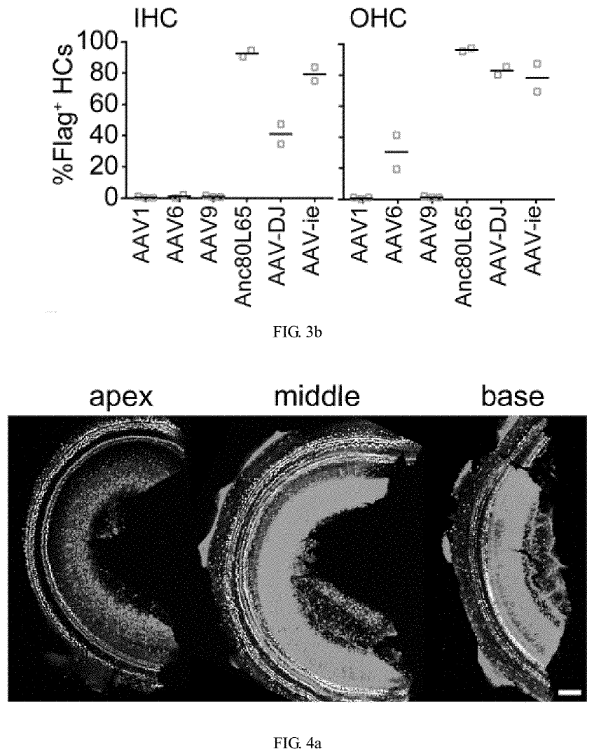

As shown in FIGS. 3a-3b, magenta represents the supporting cells, green represents the green fluorescent protein mNeonGreen expressed in the AAV-introduced cells, and FIG. 3b shows the statistical data of FIG. 3a. OHC represents outer hair cells, and IHC represents inner hair cells. The results show that the AAV variant can efficiently infect the hair cells of the in-vitro cultured mouse cochlear tissue. Compared with AAV1, AAV6, AAV9, Anc80L65 (the construction method is the same as that in Embodiment 1, the only difference is the Rep-Cap plasmid used, and the plasmid sequence is shown in SEQ ID NO. 7-10) and AAV-DJ, the variant AAV-ie has a higher infection rate to Myo7a-positive hair cells.

The above results indicate that the AAV variant AAV-ie can efficiently infect Myo7a-positive hair cells and Sox2 protein-positive supporting cells.

### Embodiment 3 AAV Variants can Efficiently Infect Various Tissue Cells in Mouse Cochlea after In-Vivo Injection

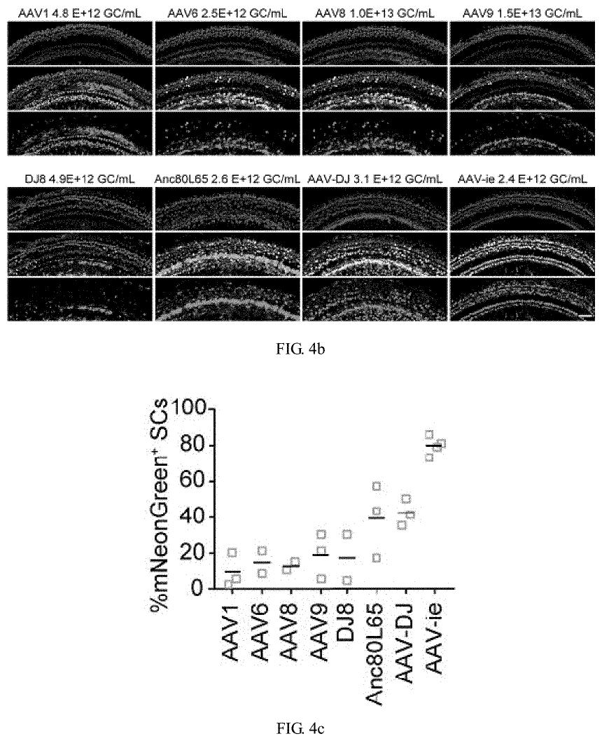

By using the cochlear round window injection technique, 1.5 μL of AAV variant viruses (the concentrations of the viruses are shown in FIG. 4) were injected into the cochlear perilymph. The specific steps are as follows: the newborn mice were anesthetized by low-temperature induced anesthesia method. P2-3 mice were placed in an ice bath for 2-3 minutes, and removed to an ice pad for subsequent surgical procedures. The operation was performed only in the left ear of each mouse, and the right ear served as a negative control. During the operation, an incision was made behind the left ear and the round window was exposed according to the relative positional relationship between the temporal bone and facial nerve. Avoid damage to the facial nerve during surgery. Next, using a micro-sampling system (Nanoliter 2000, WPI) to inject the AAV into the cochlea through the round window by a capillary glass electrode (diameter of 10 mm). The cochlea of a young mouse may hold 2 μL of AAV virus solution. The volume of the injected virus is 1-2 μL. After the operation, suturing the wound, applying the painkiller and the anti-inflammatory drug.

The mouse strain used was C57/B6. 10 days after the injection, peeling out the cochlea. Identifying the samples by immunostaining. The samples were immersed in 4% PFA for fixation, then immersed in PBS containing 10% donkey serum and 0.3% Triton X-100. After incubating at room temperature for 1 hour, adding the antibodies of Myo7a, Sox2, NeurN and the corresponding secondary antibodies. The samples were mounted with an anti-fluorescence quenching agent mounting medium, and observed with confocal microscopy.

As shown in FIGS. 4a-4c, magenta represents the Sox2-positive supporting cells, green represents the green fluorescent protein mNeonGreen expressed in the AAV-introduced cells. FIG. 4c represents the counting statistics of FIG. 4b. The result shows: 1. FIG. 4a shows the expression of green fluorescence mNeonGreen and the staining results of the supporting cell marker protein Sox2 represented by magenta in three areas of the cochlea tissue photographed in a large field of view: apex, middle and base. It can be found that AAV-ie can efficiently introduce fluorescent protein into various cells of the cochlea; 2. FIGS. 4b and 4c show that the AAV variant can efficiently infect supporting cells in the mouse cochlear tissue in vivo. Compared with AAV1, AAV6, AAV8, AAV9, DJ8, Anc80L65 and AAV-DJ, the variant AAV-ie has a higher infection rate to Sox2-positive supporting cells.

As shown in FIGS. 5a-5b, magenta represents the Myo7a-positive hair cells, green represents the green fluorescent protein mNeonGreen expressed in the AAV-introduced cells. IHC represents inner hair cells, OHC represents outer hair cells. FIG. 5b represents the counting statistics of FIG. 5a. The results show that the AAV variant AAV-ie can efficiently infect the hair cells of the mouse cochlear tissue in vivo. Compared with the parental AAV, the variant AAV-ie has a higher infection rate to Myo7a-positive hair cells.

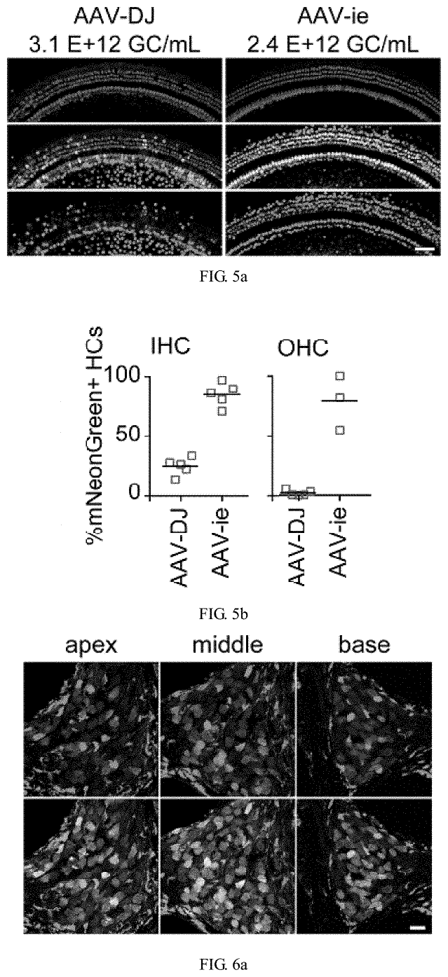

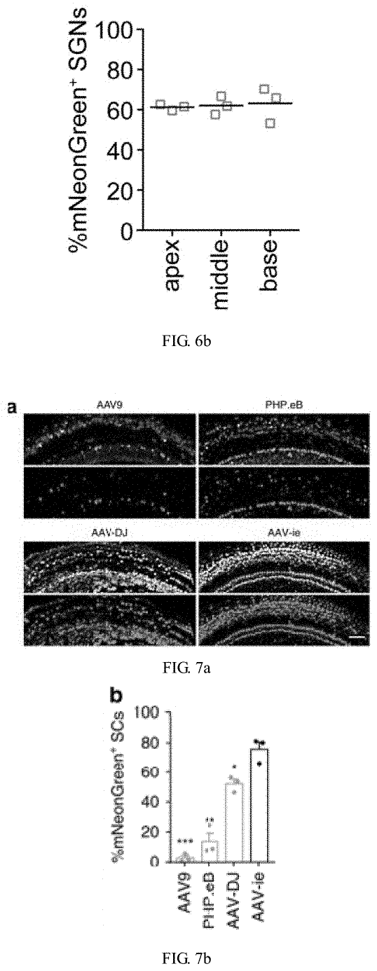

As shown in FIGS. 6a-6b, magenta represents the NeurN-positive spiral ganglion neurons, green represents the green fluorescent protein mNeonGreen expressed in the AAV-introduced cells. FIG. 6b represents the counting statistics of FIG. 6a. The results showed that in three areas of the cochlea (apex, middle and base), the AAV variant AAV-ie can efficiently infect the spiral ganglion neurons of the in-vitro cultured mouse cochlear tissue.

The above results indicate that the AAV variant using round window injection method can efficiently infect Myo7a-positive hair cells, Sox2-positive supporting cells and NeuroN-positive spiral ganglion neurons.

### Embodiment 4 AAV-ie can Infect Supporting Cells of Cochlear More Efficiently than PHP.eB

The AAV-ie is obtained by inserting the amino acid fragment DGTLAVPFK (SEQ ID NO. 1) into the capsid protein VP1 of the wild-type AAV-DJ. PHP.eB is an AAV obtained by inserting the same peptide fragment DGTLAVPFK into the capsid protein VP1 of AAV9. PHP.eB is an AAV that can cross the blood-brain barrier. AAV9, PHP.eB, AAV-DJ, and AAV-ie were injected into mouse cochlea in vivo. The results in FIG. 7 show that AAV-ie has the highest infection rate to cochlear supporting cells.

By using the cochlear round window injection technique, 1.5 μL of AAV viruses (all injected with the same amount of viruses (3.6×10⁹ GCs)) were injected into the cochlear perilymph. The specific steps are as follows: the newborn mice were anesthetized by low-temperature induced anesthesia method. P2-3 mice were placed in an ice bath for 2-3 minutes, and removed to an ice pad for subsequent surgical procedures. The operation was performed only in the left ear of each mouse, and the right ear served as a negative control. During the operation, an incision was made behind the left ear, and the round window was exposed according to the relative positional relationship between the temporal bone and facial nerve. Avoid damage to the facial nerve during surgery. Next, using a micro-sampling system (Nanoliter 2000, WPI) to inject the AAV into the cochlea through the round window by a capillary glass electrode (diameter of 10 mm). The cochlea of a young mouse may hold 2 μL of AAV virus solution. The volume of the injected virus is 1-2 μL. After the operation, suturing the wound, applying the painkiller and the anti-inflammatory drug.

The mouse strain used was C57/B6. 10 days after the injection, peeling out the cochlea. Identifying the samples by immunostaining. The samples were immersed in 4% PFA for fixation, and then immersed in PBS containing 10% donkey serum and 0.3% Triton X-100. After incubating at room temperature for 1 hour, adding the antibody of Sox2 and the corresponding secondary antibody. The samples were mounted with an anti-fluorescence quenching agent mounting medium, and observed with confocal microscopy.

As shown in FIG. 7a, magenta represents the Sox2-positive supporting cells, green represents the green fluorescent protein mNeonGreen expressed in the AAV-introduced cells. FIG. 7b represents the counting statistics of FIG. 7a. The result shows that: 1. AAV-ie can efficiently introduce fluorescent protein into Sox2-positive supporting cells; 2. FIG. 7b shows that the AAV variant can efficiently infect supporting cells in the mouse cochlear tissue in vivo. Compared with AAV9, PHP.eB and AAV-DJ, the variant AAV-ie has a higher infection rate to Sox2-positive supporting cells.

The above results indicate that the AAV-ie obtained by inserting the amino acid fragment DGTLAVPFK into AAV-DJ can infect cochlear supporting cells more effectively than the PHP.eB obtained by inserting DGTLAVPFK into AAV9.

### Embodiment 5 In-Vivo Injection of Atoh1-Carrying AAV-ie into the Cochlea Causes the Regeneration of a Large Number of Hair Cells

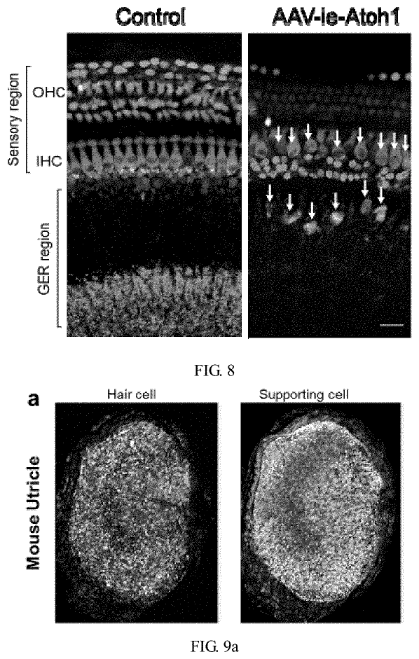

By using the cochlear round window injection technique, 1.5 μL of AAV-ie-Atoh1 viruses (virus concentration of 5E+12 GC/mL) (Refer to embodiment 1 for construction method, the plasmid used was replaced by a plasmid expressing Atoh1 gene (NCBI Reference Sequence: NM_007500.5)) carrying mouse Atoh1 gene (NCBI Reference Sequence: NM_007500.5) were injected into the cochlear perilymph. For the specific method, please refer to Embodiment 3. The mouse strain used was C57/B6. The mice used were young mice 2-3 days after birth. 10 days after the injection, peeling out the cochlea. Identifying the samples by immunostaining. The samples were immersed in 4% PFA for fixation, and then immersed in PBS containing 10% donkey serum and 0.3% Triton X-100. After incubating at room temperature for 1 hour, adding the antibodies of Myo7a, Sox2 and the corresponding secondary antibodies. The samples were mounted with an anti-fluorescence quenching agent mounting medium, and observed with confocal microscopy. The results are shown in FIG. 8, magenta represents the immunostaining of the hair cell marker gene Myo7a, green represents the immunostaining of the supporting cell marker gene Sox2, and white arrows represent ectopically regenerated hair cells.

The above results show that AAV-ie-Atoh1 virus can significantly regenerate hair cells in the sensory region and GER region after introducing Atoh1 gene into the supporting cells.

### Embodiment 6 AAV-ie can Efficiently Infect Mouse and Human Utricle Cells

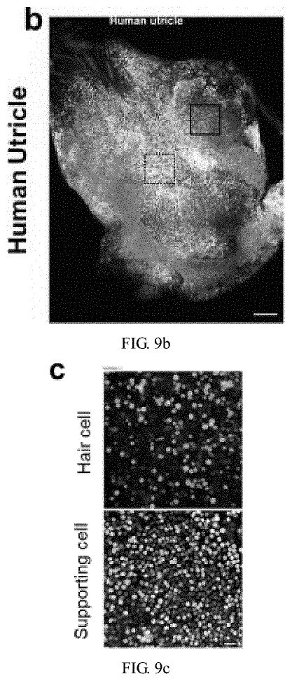

The utricle is an organ in the inner ear that senses gravity and maintains balance. By using the cochlear round window injection technique, 1.5 μL of AAV-ie viruses (concentration of the virus is 6E+12 GC/mL) were injected into the cochlear perilymph. For the specific method, please refer to Embodiment 3. The mouse strain used was C57/B6. The mice used were young mice 2-3 days after birth. 10 days after the injection, peeling out the utricle. Identifying the samples by immunostaining. The samples were immersed in 4% PFA for fixation, and then immersed in PBS containing 10% donkey serum and 0.3% Triton X-100. After incubating at room temperature for 1 hour, adding the antibodies of Myo7a, Sox2 and the corresponding secondary antibodies. The samples were mounted with an anti-fluorescence quenching agent mounting medium, and observed with confocal microscopy. As shown in FIG. 9a, magenta represents the hair cell marker protein Myo7a and the supporting cell marker protein Sox2, respectively, green represents the green fluorescent protein mNeonGreen delivered by AAV-ie. It can be found that AAV-ie can efficiently infect the hair cells and supporting cells of the utricle of the mice.

Taking the utricle of human, and infecting with 5×10¹⁰ GCs of AAV-ie virus. After 7 days, the human samples were immersed in 4% PFA for fixation, and then immersed in PBS containing 10% donkey serum and 0.3% Triton X-100. After incubating at room temperature for 1 hour, adding the antibodies of Myo7a, Sox2 and the corresponding secondary antibodies. The samples were mounted with an anti-fluorescence quenching agent mounting medium, and observed with confocal microscopy. As shown in FIGS. 9b-9c, green represents the green fluorescent protein mNeonGreen delivered by AAV-ie. Red represents the hair cell marker protein Myo7a, and magenta represents the supporting cell marker protein Sox2. The result shows that AAV-ie is also capable of efficiently infect the hair cells and supporting cells of the utricle of human.

### Embodiment 7 Safety Study of AAV Variant

By using the cochlear round window injection technique, 1.5 μL of AAV-ie were injected into the cochlear perilymph. The specific steps are as follows: the newborn mice were anesthetized by low-temperature induced anesthesia method. P2-3 mice were placed in ice bath for 2-3 minutes, and removed to an ice pad for subsequent surgical procedures. The operation was performed only in the left ear of each mouse, and the right ear served as a negative control. During the operation, an incision was made behind the left ear, and the round window was exposed according to the relative positional relationship between the temporal bone and facial nerve. Avoid damage to the facial nerve during surgery. Next, using a micro-sampling system (Nanoliter 2000, WPI) to inject the AAV into the cochlea through the round window by a capillary glass electrode (diameter of 10 mm). The cochlea of a young mouse may hold 2 μL of AAV virus solution. The volume of the injected virus is 1-2 μL. After the operation, suturing the wound, applying the painkiller and the anti-inflammatory drug.

The mouse strain used was C57/B6. 30 days after injection, the hearing of the mice was measured using ABR technology, and then the virus-injected cochlea and the non-virus-injected cochlea peeled out. Dehydrating the cochlea by gradient, preparing the sample for scanning electron microscope (SEM). Then observing the morphology of the cochlea by the scanning electron microscope.

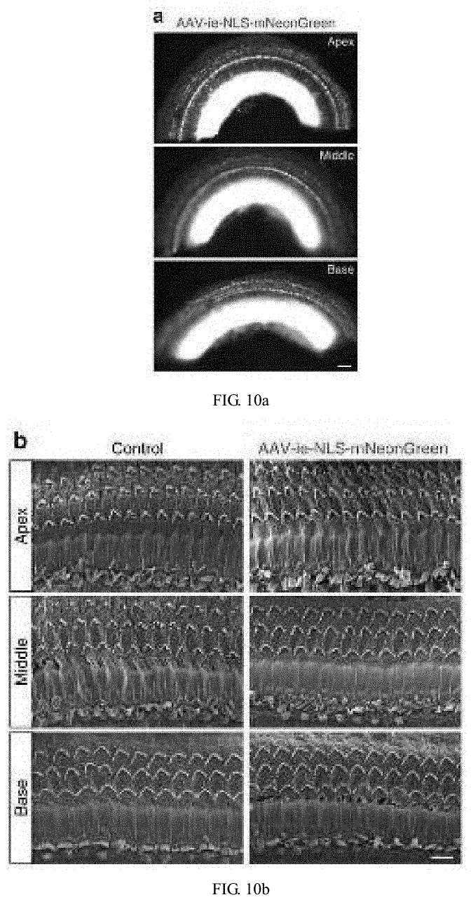

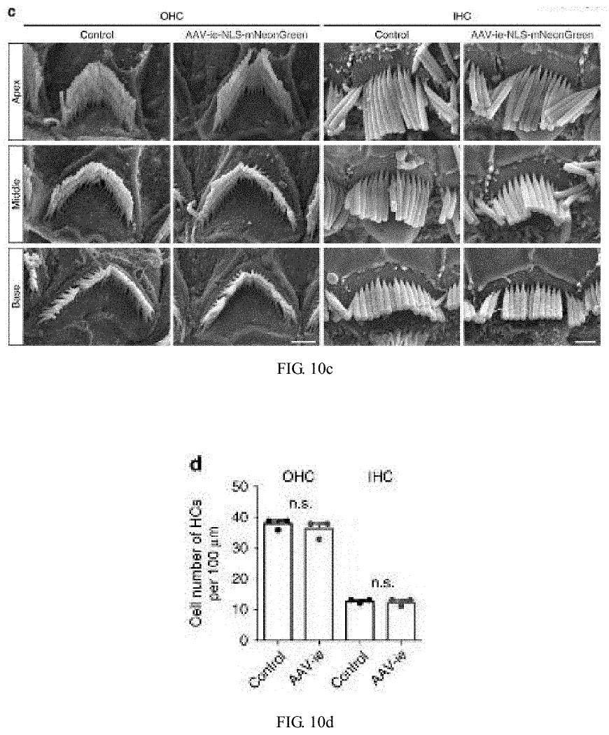

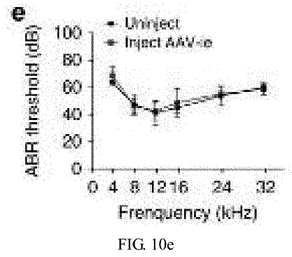

As shown in FIG. 10a, after the cochlea was peeled out, the fluorescence microscope observations found out that, the AAV-ie-injected cochlea did fluoresce due to virus infection. The SEM observation of FIG. 10b shows that there was no loss of cells in AAV-ie-infected cochlea. FIG. 10c shows the enlarged view of FIG. 10b, indicating there was no damage to the details of the cell surface. FIG. 10d shows the statistical results of the cells in FIG. 10b. Compared with the Control, AAV-ie infection did not result in cell loss. The result of ABR audiometry technology in FIG. 10e shows that AAV-ie injection does not affect the hearing of mice.

The above results indicate that AAV-ie is a safe viral vector and would not affect the normal function of the cochlea.

The above embodiments are intended to illustrate the disclosed embodiments of the present disclosure and are not understood as restrictions on the present disclosure. In addition, various modifications of the present disclosure, as well as variations of the methods and compositions of the disclosure, will be apparent to those skilled in the art without departing from the scope of the disclosure. While the disclosure has been described in detail in connection with various specific preferred embodiments thereof, however, it should be understood that the present invention should not be limited to these specific embodiments. In fact, various modifications to the disclosure as apparent to those skilled in the art are intended to be included within the scope of the disclosure.

1. A capsid protein VP1 of a variant adeno-associated virus AAV-ie, wherein an amino acid fragment is inserted between N589 and R590 of a capsid protein VP1 of a wild-type AAV-DJ, and an amino acid sequence of the amino acid fragment is shown in SEQ ID NO. 1.

2. The capsid protein VP1 of the variant adeno-associated virus AAV-ie according to claim 1, wherein an amino acid sequence of the capsid protein VP1 of the variant adeno-associated virus AAV-ie is shown in SEQ ID NO. 4.

3. An isolated nucleic acid, comprising a nucleotide sequence encoding the capsid protein VP1 of the variant adeno-associated virus AAV-ie according to claim 1.

4. A construct, comprising the isolated nucleic acid according to claim 3.

5. A host cell, comprising the construct according to claim 4 or incorporating an exogenous isolated nucleic acid according to claim 3 in a genome.

6. A variant adeno-associated virus AAV-ie, comprising the capsid protein VP1 of the variant adeno-associated virus AAV-ie according to claim 1.

7. The variant adeno-associated virus AAV-ie according to claim 6, further comprising a heterologous nucleotide sequence encoding a target product.

8. The variant adeno-associated virus AAV-ie according to claim 6, wherein the target product is a nucleic acid or a protein, and the nucleic acid is preferably selected from small guide RNA or interfering RNA.

9. A pharmaceutical composition, comprising the variant adeno-associated virus AAV-ie according to claim 6 and pharmaceutically acceptable excipients.

10. Use of the variant adeno-associated virus AAV-ie according to claim 8 for delivering a target product to hair cells and/or supporting cells of an individual.

11. (canceled)

12. Use of the variant adeno-associated virus AAV-ie according to claim 8 in preparation of drugs for treatment of a hearing impairment disease caused by cochlear injury in an individual.

13. (canceled)

14. (canceled)

15. A pharmaceutical composition, comprising the variant adeno-associated virus AAV-ie according to claim 7 and pharmaceutically acceptable excipients.

16. A pharmaceutical composition, comprising the variant adeno-associated virus AAV-ie according to claim 8 and pharmaceutically acceptable excipients.

17. Use of the variant adeno-associated virus AAV-ie according to claim 6 for delivering a target product to hair cells and/or supporting cells of an individual.

18. Use of the variant adeno-associated virus AAV-ie according to claim 7 for delivering a target product to hair cells and/or supporting cells of an individual.

19. Use of the variant adeno-associated virus AAV-ie according to claim 6 in preparation of drugs for treatment of a hearing impairment disease caused by cochlear injury in an individual.

20. Use of the variant adeno-associated virus AAV-ie according to claim 7 in preparation of drugs for treatment of a hearing impairment disease caused by cochlear injury in an individual.

21. The use according to claim 10, wherein the target product is a nucleic acid or a protein, and the nucleic acid is preferably selected from small guide RNA or interfering RNA.

22. The use according to claim 12, wherein the hearing impairment disease is a disease related to genetic defects, environmental damage or aging.

23. The use according to claim 12, wherein the hearing impairment disease is a disease related to cell damage.
